(19) United States
(12) Patent Application Publication
(10) Pub. No.: US 2021/0219570 A1
(43) Pub. Date: Jul. 22, 2021

(54) CIRCULAR INTEGRATED ECOLOGICAL BREEDING AND PLANTING SYSTEM

(71) Applicant: RONG SHIN JONG CO., LTD., Zhubei City (TW)

(72) Inventor: YI JU CHUNG, ZHUBEI CITY (TW)

(21) Appl. No.: 17/149,076

(22) Filed: Jan. 14, 2021

(30) Foreign Application Priority Data
Jan. 20, 2020 (TW) 109200804

(51) Int. Cl. A23K 10/37; A01G 9/14; A01G 33/00; A01C 21/00; A01K 61/10; A01C 3/00; A01K 29/00; A01K 67/033; A01K 61/59; A01K 61/50; A01K 45/00; A23K 10/22; A23K 10/26; A23K 50/80; A23K 50/90; A23K 50/75; A23N 17/00

(52) U.S. Cl. CPC A23K 10/37; A23N 17/007; A01G 33/00; A01C 21/00; A01K 61/10; A01C 3/00; A01K 29/005; A01K 67/033; A01K 61/59; A01K 61/50; A01K 67/0332; A01K 45/00; A23K 10/22; A23K 10/26; A23K 50/80; A23K 50/90; A23K 50/75; A01G 9/14

(57) ABSTRACT

A circular integrated ecological breeding and planting system including a plurality of units capable of growing vegetables, producing worms, breeding and raising chickens, and growing seaweed is provided to collectively construct a simplified simulated food. It not only unifies the lower and upper ecological food chains, but also minimizes pollution and undesirable waste. The system is particularly suitable for enclosed and intensive production within a single enclosed building to greatly reduce the production cost. This proposed eco-agricultural model has applications beyond its current configuration and can be applied to grow other plants, and to breed and raise other animals in the future.

This application claims priority of Application No. 109200804 filed in Taiwan on 20 Jan. 2020 under 35 U.S.C. § 119; the entire contents of all of which are hereby incorporated by reference.

### BACKGROUND OF THE INVENTION

### Field of the Invention

The present invention relates to a planting and breeding system, in particular, to an ecological circular integrated breeding and planting system, with the integration of animals, plants, microorganisms, soil and water.

### Description of the Prior Art

As the climate is getting warmer and environment pollution issues intensify, environmental protection and food topics have attracted much attention. Specifically, the greenhouse effect is a cause of global warming which deteriorates the climate. As the climate is getting worse, various large-scale natural disasters such as tsunamis, blizzards, heat waves, or severe temperature variations may have led to a significant reduction in arable farming land, and therefore food production has become increasingly unstable. Further, the extreme phenomena such as droughts and floods in various places may cause a sudden drop in food production (e.g. planting and breeding). Furthermore, total population increase and resource depletion leads the situation getting even worse. In addition, according to an UN estimation, a global food shortage may occur within the next 10 years, and hunger will become more common.

On the other hand, the food production may also cause environmental damage. Taking livestock farming as an example, not only large amounts of cereal feed or natural grass are consumed in the process of raising cattle, but also large amounts of methane and feces are produced. Therefore, how to take the environmental protection into account while increasing food production becomes a key issue. Further, the food safety issues have been receiving more and more attention recently. If the production of high-quality organic ingredients can be increased while ensuring food safety, it will be of great help to the stable development of the social economy.

In this regard, many experts and scholars have put forward suggestions for indoor planting and breeding, such as setting breeding and planting bases inside buildings or large cruise ships, or setting breeding and planting farms above the sea level, under the sea surface or underground. Although the planting of fruits and vegetables in greenhouses and the indoor raising of poultry have been practiced for a long time, the output is mainly raised by mere the control of simple environmental parameters, whereas other factors, such as environmental sustainability and waste disposal, are not jointly concerned.

Moreover, as the resource depletion is getting worse, and human population is getting denser, people also target extraterrestrial developments such as the construction of spaceships, earth orbit bases, space bases, terrestrial planets, etc. When the time has come, it will be necessary to use a high-efficiency indoor produce base as a basis for sustainable development.

In summary, the high-efficiency and intensive indoor agriculture is indeed a future development trend. The high-efficiency and intensive indoor agriculture is a solution for the areas with scarce resources and extreme climate, or a solution to overcome the food shortages due to rising population. To be simply served as a foundational template, the circular integrated ecological breeding and planting system is the key to high-efficiency and intensive indoor agricultural practices in the future. In view of this, the present invention proposes a circular integrated ecological breading and planting system, which is a future eco-agriculture model integrating of social benefit, economic benefit and ecological environment protection, in order to lay a foundation for future breeding and planting base. The detailed architectures and embodiments are described as follows.

### SUMMARY OF THE INVENTION

A primary objective of the present invention is to provide a circular integrated ecological breading and planting system applied in an enclosed building, which may greatly increase the overall capacity without being affected by geographical, environmental or weather/seasonal factors, and may become a high-efficiency and intensive combined agriculture, so that the economic value of produce per unit area is maximized.

Another objective of the present invention is to provide a circular integrated ecological breading and planting system, which cooperates with technological simulation to simplify the concept of ecological food chain, and makes full use of by-products or waste generated in each link for reusing and recycling them. It is a future eco-agriculture model that integrates social-benefit, economic benefit and ecological environment protection, and may also be used as the basis for future breeding and planting bases.

To achieve above objectives, the present invention discloses a circular integrated ecological breading and planting system which includes a planting and breeding system, and a monitoring module. The planting and breeding system includes a plantation greenhouse, a feed processing field, a seafood farm, an insect farm, a livestock farm, and a seaweed farm, wherein the plantation greenhouse is used for growing at least one plant to obtain crops and plant waste; the feed processing field may be used for receiving and processing the plant waste into a feed, fertilizer that may be used by the plantation greenhouse to provide the plant with fertilization and residues; the seafood farm is used for breading seafood by the feed to produce a seafood product and seafood waste that may be processed by the feed processing field; the insect farm is used for breading, by the feed, at least one insect that may be processed into a high protein insect powder, the high protein insect powder being mixed into the feed or the fertilizer for use; the livestock farm is used for breading, by the feed, at least one animal that may be provided to produce at least one animal food and an animal waste, wherein the animal waste is processed by the feed processing field; the seaweed farm may receive the plant waste, the seafood waste, and the animal waste as raw material additives for cultivating the seaweed, wherein in addition to providing additional economic value, the seaweed product may be used as another source of feed, producing large amounts of high-quality oxygen, and the waste produced may be used as a source of biogas and fertilizer.

The monitoring module may guarantee the best growing environment in the planting and breeding zone through 24-7 comprehensive monitoring, management, filtration, disinfection, etc. For example, the monitoring module may monitor and deploy various breeding and planting states of the breeding and planting system, including the temperature, humidity, illuminated time, constituent elements in air, respective concentration of the constituent elements in air, bacterial quota in air, water quality, water temperature, water pH, salinity of water, constituent ratio of bacteria and other elements in water.

Therefore, through the cooperation of these links, the products, by-products and waste may be effectively applied, which not only meet the needs of environmental protection without causing pollution, but also may be used effectively, so that the economic value of produce per unit area is maximized to reach the circular simulation ecological food chain. Therefore, the goal of “scientific planting and breeding” with the largest output per unit area can be achieved on a smallest land.

The following detailed descriptions will be made with specific examples and accompanying drawings to make it easier to understand the purpose, technical content, characteristics, and effects of the disclosure.

### BRIEF DESCRIPTION OF THE DRAWINGS

FIG. 1 is a view of a circular integrated ecological breading and planting system provided by an embodiment of the present invention;

FIG. 2 is a view of functions of a plantation greenhouse of a circular integrated ecological breading and planting system provided by an embodiment of the present invention;

FIG. 3 is a view of functions of a feed processing field of a circular integrated ecological breading and planting system provided by an embodiment of the present invention;

FIG. 4 is a view of functions of a seafood farm of a circular integrated ecological breading and planting system provided by an embodiment of the present invention;

FIG. 5 is a view of functions of an insect farm of a circular integrated ecological breading and planting system provided by an embodiment of the present invention;

FIG. 6 is a view of functions of a livestock farm of a circular integrated ecological breading and planting system provided by an embodiment of the present invention;

FIG. 7 is a view of functions of a seaweed farm of a circular integrated ecological breading and planting system provided by an embodiment of the present invention.

### DESCRIPTION OF THE PREFERRED EMBODIMENTS

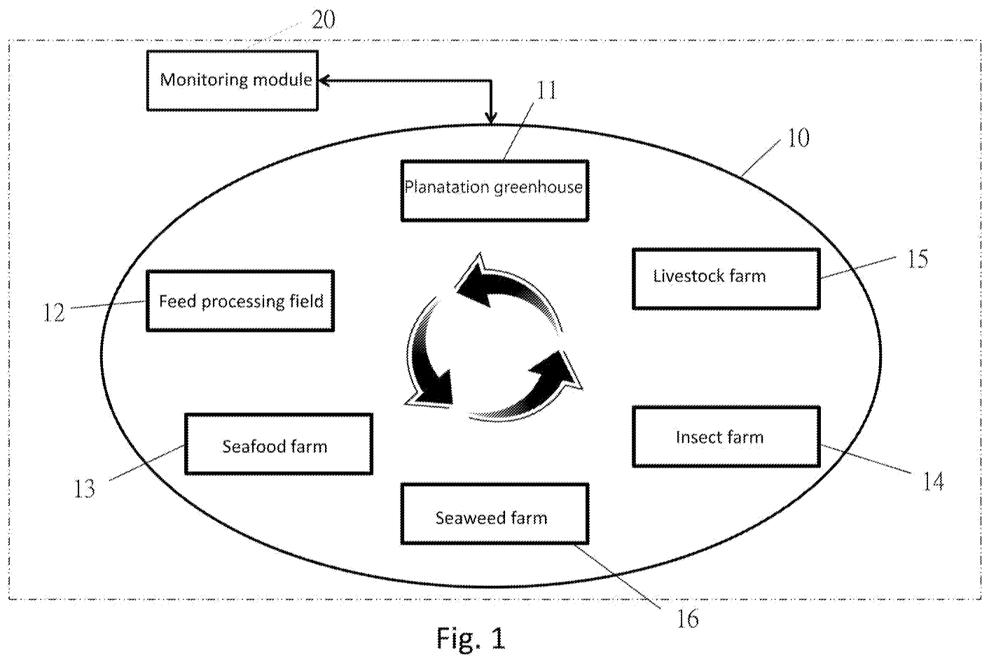

With reference to FIG. 1, a view of a circular integrated ecological breading and planting system provided by an embodiment of the present invention is shown.

In order to achieve a high-efficiency and intensive agriculture with almost zero pollution, the first concept of ecosphere and food chain is considered to use the system of the present invention to create a simulation of technology to simplify the ecological food chain, which may effectively reuse and recycle the by-products and waste generated in each stage, and may maximize the economic value of produce per unit area, so as to constitute a completely enclosed circular breeding and planting system with the simulation of technology to simplify the ecological food chain.

Therefore, as shown in FIG. 1, the present invention discloses a circular integrated ecological breading and planting system applied to an enclosed building. The enclosed building may be a variety of building interiors, large cruise ships, places on or below the sea level, or underground breeding and planting farms and other breeding bases, or also future breeding bases such as spaceships, earth orbit bases, space stations, terrestrial planets, etc. Specifically, the circular integrated ecological breading and planting system includes a planting and breeding system 10, and a monitoring module 20, wherein the planting and breeding system 10 includes a plantation greenhouse 11, a feed processing field 12, a seafood farm 13, an insect farm 14, a livestock farm 15, and a seaweed farm 16. Among them, the plantation greenhouse 11 is used for growing plants to obtain crops and plant waste. The feed processing field 12 may be used for processing the plant waste to generate feed, fertilizer and residues. The fertilizer may be used by the plantation greenhouse 11 to provide the vegetable and fruit plant with fertilization. The seafood farm 13 is used for breading seafood by the feed to produce a seafood product and seafood waste. The seafood waste may be performed with circulation processing by the feed processing field 12. The insect farm 14 is used for breading various insects by the feed, wherein the insects may be processed into a high protein insect powder. The high protein insect powder can be mixed into the feed or fertilizer for use in husbandry or breeding. The livestock farm 15 is used for breading various animals by the feed. The animals may provide eggs, dairy products, and the animals can be meat materials and other biological waste. The biological waste can be processed by the feed processing field 12. The seaweed farm 16 may mainly breed seaweed as the feed or the fertilizer, and the seaweed can additionally provide oxygen or be an energy source. By monitoring and deploying various breeding and planting states of the breeding and planting system 10, e.g. the temperature, humidity, illuminated time, constituent elements in air, respective concentration of the constituent elements in air, bacterial quota in air, water quality, water temperature, water pH, salinity in water, constituent ratio of bacteria and elements in water, the monitoring module 20 may guarantee the best growing environment in the planting and breeding zone through 24-7 comprehensive monitoring, management, filtration, disinfection, etc. Although the various units as disclosed above are recorded as treatment plants or farms, etc., it is more than just a factory or plant concept, but a module, unit, machine, or system to achieve the desired function. The following will further elaborate on each part of the circular integrated ecological breading and planting system.

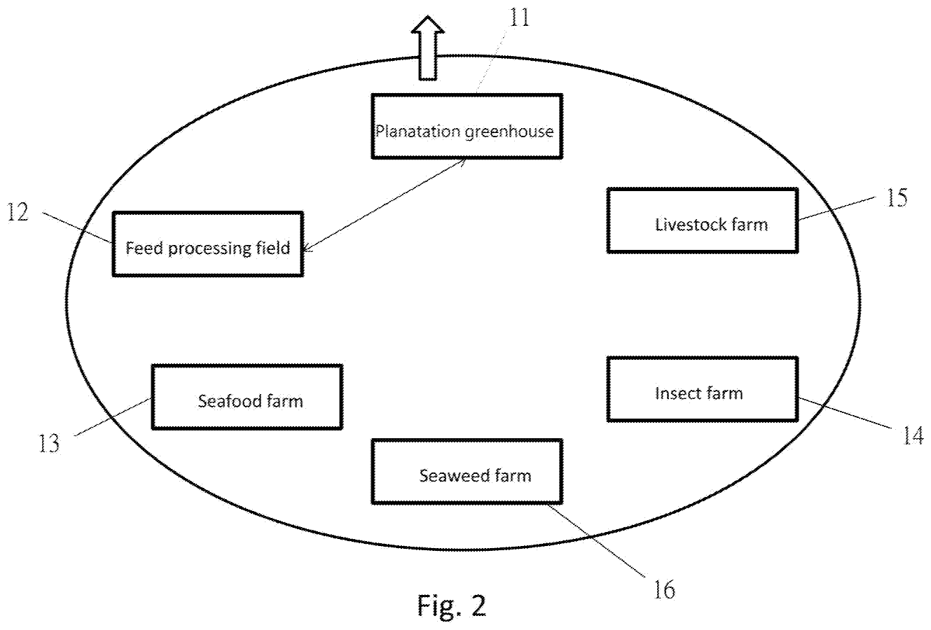

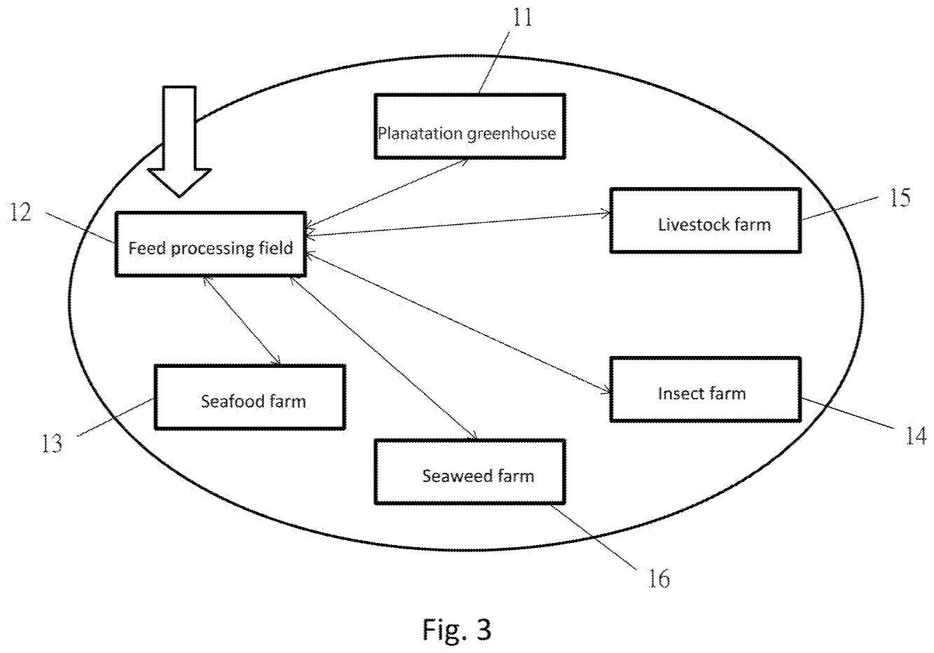

As shown in FIG. 2, the plantation greenhouse 11 is mainly used for planting vegetables, fruits, fungi and other edible or available plants, and the main products are agricultural crops of various crops such as vegetables, fruits and fungi. The corresponding plant waste generated during the planting process may contain such as inedible parts of the aforementioned plants or waste during planting, fallen leaves, peels or wood chips, etc. The plant waste may be processed by the feed processing field 12 into feed, fertilizer and residues. Certainly, in addition to the aforementioned edible plants, it may also be various plants required for fertilization and animal feeding, and is not particularly limited here. Furthermore, planting various plants may optimize the overall air and effectively maintain clean air in the breeding and planting base. With continuous reference to FIG. 3, in addition to receiving the plant waste from the aforementioned plantation greenhouse 11, the feed processing field 12 may, according to the actual situation and needs, use high-temperature disinfection, sterilization, distillation, fermentation and other means to process and produce the feed required for the seafood farm 13, the insect farm 14 and the livestock farm 15, or to process and produce the fertilizer required for the plantation greenhouse 11 or seaweed farm 16. The source of raw materials may come from purchased produce waste for mixing, such as wheat straw, rice straw, corn bran, beer residues, soybean meal, corn, rice or other wheat and other produce waste, so that the nutrients contained in its feed or fertilizer are more complete. Therefore, in addition to processing and producing feed and fertilizer, the feed processing field 12 makes the remaining unusable parts become residues. Apart from the aforementioned plant waste, other units also produce corresponding waste that may be supplied as a source of other raw materials. This section will be described in detail later.

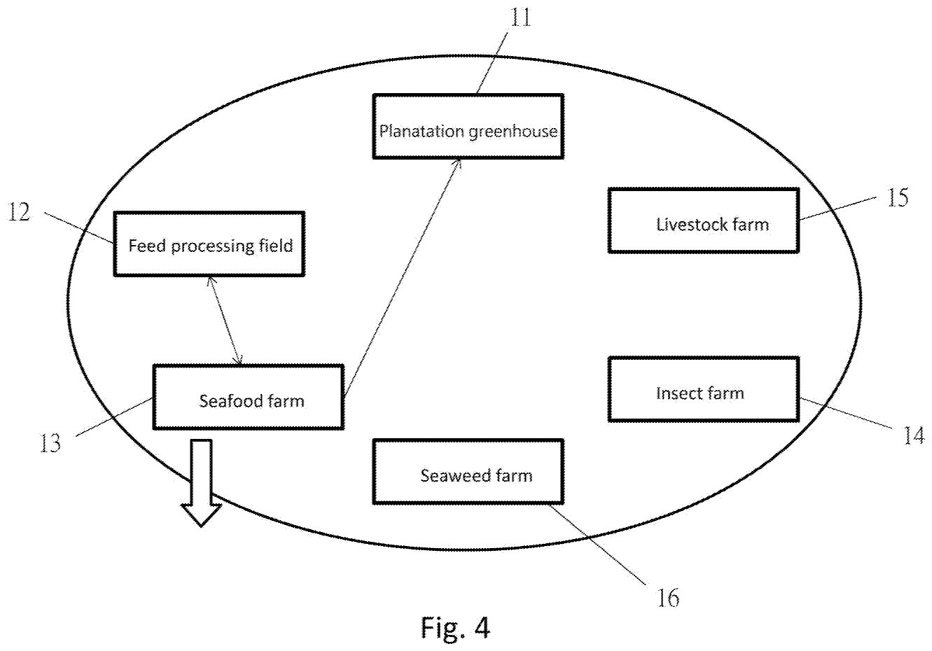

As shown in FIG. 4, the seafood bred in the seafood farm 13 comprises fish, crustaceans and shellfish. In addition to producing high-quality seafood product of various types of fish, crustaceans and shellfish, some seafood waste are also generated, including fish scales, blood, bones, internal organs, etc. The crustaceans and shellfish contain their crustaceans or shells. All the aforementioned materials may be transported to the feed processing field 12 for processing. Furthermore, crustaceans (such as shrimps) or shellfish may help clean and optimize the water quality. At the same time, the purified water quality should be used for irrigation in the plantation greenhouse 11, or as an application of the concept of fish and vegetable symbiosis. On the other hand, proper control on the environment and temperature in the enclosed building may greatly increase the economic benefit of farming, while reducing the possibility of disease transmission, thereby avoiding infection problems for consumers.

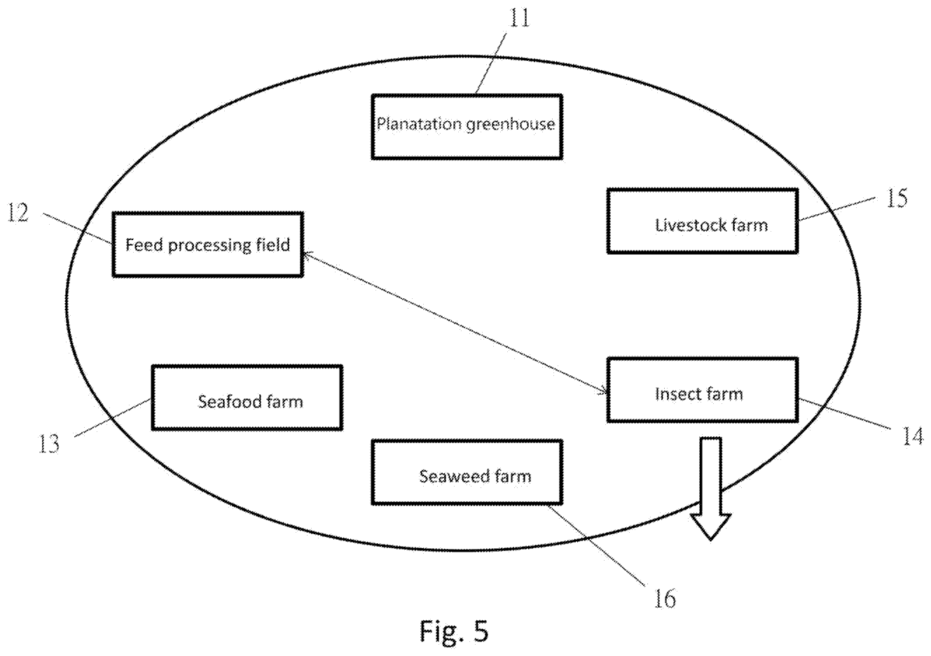

With continuous reference to FIG. 5, the insect farm 14 may be used to feed insects such as mealworms, earthworms, maggots, etc., which may be processed into high protein insect powder, and the high protein insect powder may be mixed into the feed or fertilizer or sold as a terminal product.

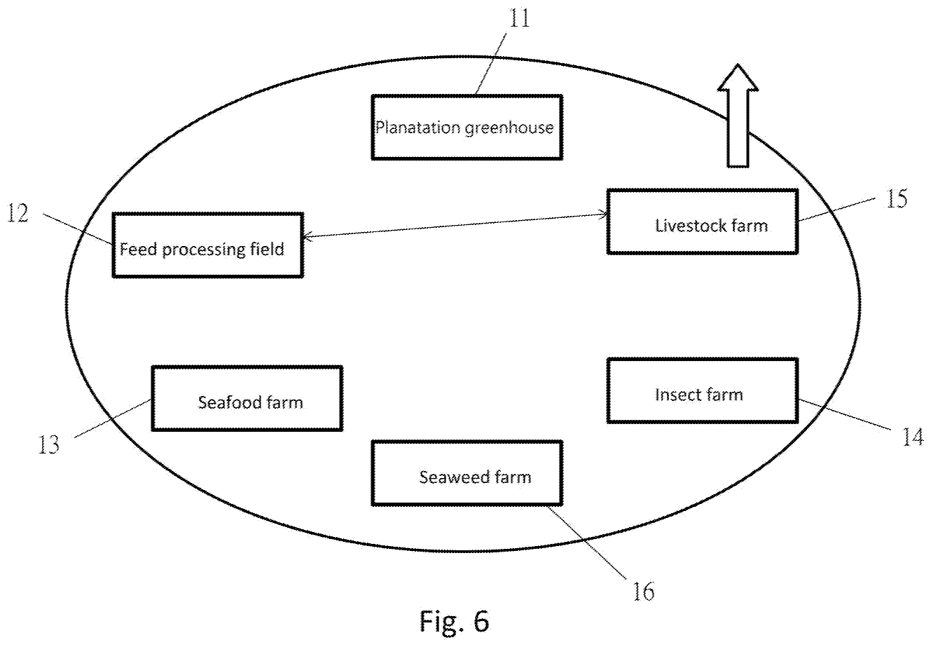

As shown in FIG. 6, the livestock farm 15 is mainly used for raising livestock, and its feed source may be produced by the feed processing field 12. The livestock farm 15 may produce various animal foods, such as meat, eggs, offal or other edible parts, etc. As for the remaining non-edible waste, such as hair, blood, grease, bones, inedible parts and internal organs, feces, etc., they may also be transported to the feed processing field 12 for being processed into the feed and fertilizer. This closed-type feeding method may reduce the spread of various infectious diseases such as chicken fever, swine fever, etc., thereby improving the economic efficiency of production and effectively carrying out disease control.

The source of the feed and fertilizer processed by the above feed processing field 12 is a mixed type, and at the same time, the formula for mixing may be adjusted according to the demand. For example, the animal manure may be used as the fertilizer after fermentation, mixed with high-protein insect meal to be used as the feed for the seafood farm 13, or processed separately as the feed for the insect farm 14.

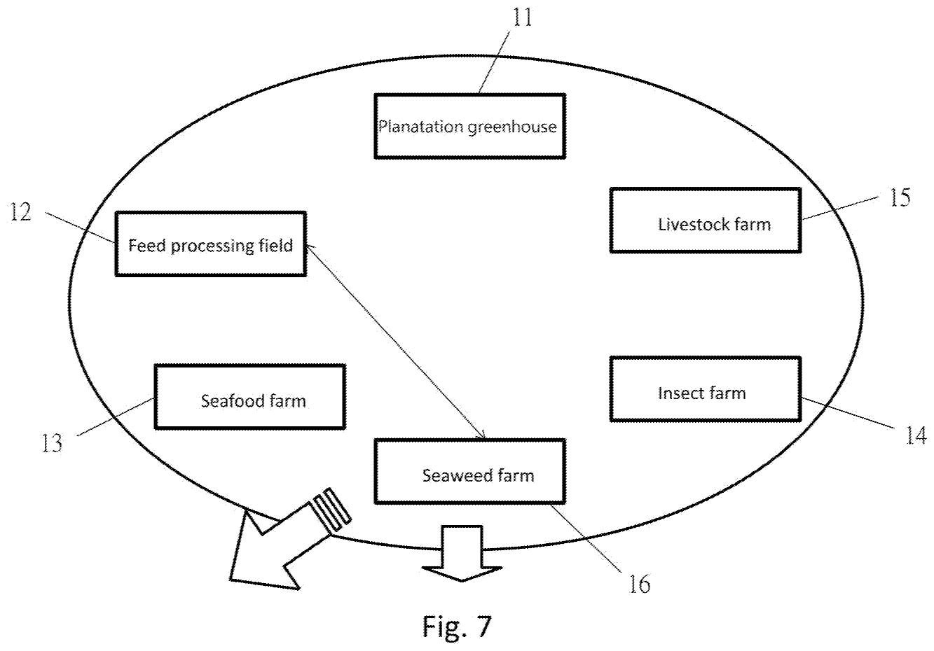

As shown in FIG. 7, the seaweed farm 16 may produce biogas slurry by fermentation, which may be combined with animal waste (such as feces) and seafood waste (such as shellfish, crustacean shells and crustaceans) to be processed into raw materials needed in the feed processing field 12 for cultivating seaweed. Meanwhile, the filtering system in the building can help supply the air and water required by the seaweed farm 16. The seaweed cultivated in the seaweed farm 16 not only produces a lot of oxygen, but also may be directly exported or processed into various nutritional supplements such as seaweed oil and seaweed tablets, or be used as a source of feed or fertilizer produced by the feed processing field 12. As for the remaining residues, they can be used as a source of compost or may be transported to the feed processing field 12 for other residues (such as plant residues or animal waste, etc.) for fermentation to produce biogas, which may be used for heating or power generation of the entire system.

With reference to FIG. 1, by using the monitoring module to monitor and deploy various breeding and planting states of the breeding and planting system 10 (including temperature, humidity, illuminated time, constituent elements in air, respective concentration of the constituent elements in air, bacterial quota in air, water quality, water temperature, water pH, salinity in water, constituent ratio of bacteria and elements in water), the monitoring module 20 may guarantee the best growing environment in the planting and breeding zone through 24-7 comprehensive monitoring, management, filtration, disinfection, etc., so as to ensure the optimal production environment and maximum output of aseptic cultivation. At the same time, through this comprehensive monitoring and closed breeding and planting environment, strict control over various raw materials and resources is also conducive to increasing the output of high-quality organic food ingredients, which is very helpful to the stabilization of the world economic development.

In summary, the circular integrated ecological breading and planting system of the present invention is simplified and constructed with the concept of technological simulation to simplify the ecological food chain, which may effectively control the source of breeding and planting of the entire system and control the safety and reliability of the entire food production. In addition, the various products, by-products and waste generated in the process may be effectively interactively applied to achieve almost zero environmental pollution. Meanwhile, the closed-type management may be monitored 24-7 and may be effectively adjusted to greatly increase the overall capacity without being affected by geographical, environmental; and weather/seasonal factors, and may become a high-efficiency and intensive combined agriculture, so that the economic value of produce per unit area is maximized. Further, the present invention may cooperate with the concept of ecological food chain and make full use of by-products or waste generated in each link for reusing and recycling. This future eco-agriculture model is not only eco-friendly but also economically beneficial, and may function as the basis for the future breeding and planting base.

The above-mentioned embodiments are only to illustrate the technical ideas and features of the present invention, and its purpose is to enable those who are familiar with this skill to understand the content of the present invention and implement it accordingly. That is to say, any changes or modifications made in accordance with the spirit of the present invention should still be covered by the patent scope of the present invention.

What is claimed is:

1. A circular integrated ecological breeding and planting system, applied in a closed building space, comprising:
a breeding and planting system, comprising:
a plantation greenhouse, for growing at least one plant to obtain agricultural crops and plant waste;
a feed processing field, for receiving and processing the plant waste into feed, fertilizer, and residues, wherein the fertilizer is to be used by the plantation greenhouse to provide the plant with fertilization;
a seafood farm, for breading seafood by the feed to produce a seafood product and seafood waste that be processed by the feed processing field;
an insect farm, for breading at least one insect by the feed, wherein the insect is to be processed into a high protein insect powder, to be mixed into the feed or the fertilizer;
a livestock farm, for breading at least one animal by the feed, wherein the animal is provided to produce at least one animal food and animal waste, and the animal waste is processed by the feed processing field; and
a seaweed farm, for providing a seaweed; and
a monitoring module, for monitoring and deploying a breeding and planting state of the breeding and planting system.

2. The circular integrated ecological breeding and planting system according to claim 1, wherein the plant grown by the plantation greenhouse comprises vegetables, fruits and fungi.

3. The circular integrated ecological breeding and planting system according to claim 1, wherein the feed processing field further purchase produce waste to be mixed and processed with the plant waste.

4. The circular integrated ecological breeding and planting system according to claim 1, wherein the seaweed farm receives the plant waste, the seafood waste, and the animal waste as raw material additives for cultivating the seaweed.

5. The circular integrated ecological breeding and planting system according to claim 1, wherein the seafood in the seafood farm comprises fish, crustaceans and shellfish.

6. The circular integrated ecological breeding and planting system according to claim 1, wherein the insect in the insect farm comprises a mealworm, an earthworm or a maggot.

7. The circular integrated ecological breeding and planting system according to claim 1, wherein the animal in the livestock farm comprises poultries and livestock.

8. The circular integrated ecological breeding and planting system according to claim 1, wherein the breeding and planting state of the breeding and planting system monitored and deployed by the monitoring module comprises temperature, humidity, illuminated time, constituent elements in air, respective concentration of the constituent elements in air bacterial quota in air, water quality, water temperature, water pH, salinity in water, constituent ratio of bacteria and elements in water.
